(19) United States
(12) Patent Application Publication
(10) Pub. No.: US 2019/0338234 A1
(43) Pub. Date: Nov. 7, 2019

(54) CELL TRAPPING FILTER, CELL TRAPPING DEVICE, CELL TRAPPING METHOD, CELL OBSERVATION METHOD, AND CELL CULTURING METHOD

(71) Applicant: HITACHI CHEMICAL COMPANY, LTD., Tokyo (JP)

(72) Inventors: Akiko ITOU, Chiyoda-ku, Tokyo (JP); Hisashige KANBARA, Chiyoda-ku, Tokyo (JP); Katsuya ENDOU, Chiyoda-ku, Tokyo (JP); Akio TAKAHASHI, Chiyoda-ku, Tokyo (JP)

(21) Appl. No.: 16/074,237

(22) PCT Filed: Jan. 26, 2017
(86) PCT No.: PCT/JP2017/002771

§ 371 Date: Jul. 31, 2018

(30) Foreign Application Priority Data
Feb. 1, 2016 (JP) 2016-017235

(51) Int. Cl. C12M 1/00; C12N 5/09; G01N 15/10; C25D 1/08

(52) U.S. Cl. CPC C12M 29/04; C25D 1/08; G01N 15/10; C12N 5/0693

(57) ABSTRACT

The filter 105 used in a cell trapping device includes a sheet-like body portion (base metal plating layer 5) containing nickel or copper as a main component and provided with a plurality of through-holes in the thickness direction; a palladium layer 7 containing palladium as a main component and covering the surface of the body portion; and a gold layer 8 containing gold as a main component and covering the surface of the palladium layer.

### TECHNICAL FIELD

The present invention relates to, a cell trapping filter that can efficiently capture rare cells, including blood circulating tumor cells (CTCs), in blood, a cell trapping device, a cell trapping method, a cell observation method, and a cell culturing method.

### BACKGROUND ART

Cancer occupies the top cause of death in countries all over the world, and in Japan, over 300000 people die of cancer annually, and early detection and treatment thereof are being desired. The death of people due to cancer is mostly caused by metastasis or recurrence of the cancer. Metastasis or recurrence of cancer occurs by that cancer cells from the primary lesion move through blood or lymph vessels and settle on and invade the blood vessel wall of another organ tissue to form a micrometastasis. Such cancer cells circulating in the human body through blood or lymph vessels are called blood circulating tumor cells.

Blood contains many blood cell components such as red blood cells, white blood cells, and platelets, and the number thereof is said to be 3.5×10⁹ to 9×10⁹ in 1 mL of blood. CTCs are only several among them. In order to efficiently detect CTCs from blood cell components, it is necessary to separate blood cell components. For this purpose, it has been investigated to remove these blood cell components by applying a mechanical filtration method to concentrate cancer cells. As the filer for performing such a mechanical filtration method, it is suggested to use a metal filter. As a method for producing a metal filter, for example, a method for electroforming plating using photolithography is known (Patent Literature 1).

### CITATION LIST

### Patent Literature

Patent Literature 1: Japanese Unexamined Patent Publication No. 2011-163830

### SUMMARY OF INVENTION

### Technical Problem

In the case of trapping rare cells, such as CTCs, using a metal filter, the metal filter may elute due to a sample solution, such as blood. If the material of the metal filter is a cytotoxic component, there is a risk of also affecting the cells trapped by the filter. As a method for preventing this, although it is suggested to cover the surface of the filter by gold plating, it may be insufficient.

The present invention has been made in view of the above problems, and the object thereof is to provide a cell trapping filter capable of suitably trapping rare cells while suppressing the trapped cells from being damaged and a cell trapping device, a cell trapping method, a cell observation method, and a cell culturing method using the cell trapping filter.

### Solution to Problem

In order to achieve the above-mentioned object, the cell trapping filter according to an aspect of the present invention includes a sheet-like body portion containing nickel or copper as a main component and provided with a plurality of through-holes in the thickness direction, a palladium layer containing palladium as a main component and covering the surface of the body portion, and a gold layer containing gold as a main component and covering the surface of the palladium layer.

It is possible to prevent the body portion of the cell trapping filter from being exposed to the outside by laminating the palladium layer and the gold layer on the surface of the body portion as described above. If the body portion is exposed to the outside, the metal ions of the body portion flow out (elute), resulting in a risk of damaging the trapped cells. In contrast, since the palladium layer is disposed on the inside of the gold layer, it is possible to prevent the body portion on the inside of the palladium layer from being exposed to the outside, and it is therefore possible to suitably perform trapping of rare cells and also to prevent the trapped cells on the cell trapping filter from being damaged by flow-out of metal ions of the body portion.

Herein, the thickness of the palladium layer may be 0.1 to 1 μm.

It is possible to further prevent the flow-out (elution) of the metal ions of the body portion of the cell trapping filter by adjusting the thickness of the palladium layer to 0.1 to 1 μm as described above.

In addition, the filer can be in a form further including a hydrophobic layer formed on the surface of the gold layer by a surface treatment agent containing phospholipid in part of the skeleton.

When a hydrophobic layer formed from a surface treatment agent containing phospholipid in a main skeleton is disposed on the surface of the gold layer of the cell trapping filter as described above, it is possible to prevent rare cells and other cells from adhering to the surface of the cell trapping filter and possible to increase the trapping efficiency of rare cells.

In addition, the cell trapping device according to an aspect of the present invention comprises a housing including an introduction flow channel for introducing a sample solution into the inside and a discharge flow channel for discharging the sample solution to the outside; and the above-described cell trapping filter disposed on a flow channel between the introduction flow channel and the discharge flow channel inside the housing in such a manner that the sample passes through the through-holes.

In the above-described cell trapping device, the lamination of the palladium layer and the gold layer on the surface of the body portion of the cell trapping filter can prevent the body portion of the cell trapping filter from being exposed to the outside. Accordingly, it is possible to suitably perform trapping of rare cells and also to prevent the trapped cells on the cell trapping filter from being damaged by flow-out of metal ions of the body portion.

In addition, the cell trapping method according to an aspect of the present invention selectively traps specific cells contained in a sample solution by performing filtration with the above-described cell trapping filter.

It is possible to selectively trap specific cells, while preventing, for example, damage of the cells due to flow-out of metal ions of the body portion by performing filtration with the above-described cell trapping filter.

In addition, the cell observation method according to an aspect of the present invention selectively traps specific cells contained in a sample solution by performing filtration with the above-described cell trapping filter, fixes the trapped specific cells on the cell trapping filter with a fixing solution, and then observing the cells under an electron microscope.

In the above-described cell observation method, it is possible to suitably observe specific cells, while preventing, for example, damage of the cells due to flow-out of metal ions from the body portion of the cell trapping filter.

In addition, the cell culturing method according to an aspect of the present invention selectively traps specific cells contained in a sample solution by performing filtration with the above-described cell trapping filter and cultures the trapped specific cells on the cell trapping filter in a culture medium.

In the above-described cell culturing method, it is possible to culture specific cells on the filter, while preventing, for example, damage of the cells due to flow-out of metal ions from the body portion of the cell trapping filter.

### Advantageous Effects of Invention

According to the present invention, a cell trapping filter capable of suitably performing trapping of rare cells while suppressing damage of the trapped cells and a cell trapping device, a cell trapping method, a cell observation method, and a cell culturing method using the cell trapping filter are provided.

### BRIEF DESCRIPTION OF DRAWINGS

FIG. 1 is a diagram illustrating a schematic structure of a cell trapping device according to an embodiment.

FIG. 2 is a diagram schematically illustrating a filter according to an embodiment.

FIG. 3 is a process diagram for explaining an example of a method for producing a filter according to an embodiment.

FIG. 4 is a process diagram for explaining another example of a method for producing a filter according to an embodiment.

### DESCRIPTION OF EMBODIMENTS

Aspects for implementing the present invention will now be described in detail with reference to the attached drawings. In explanation of the drawings, identical elements are denoted by the same reference signs, and redundant explanations are omitted.

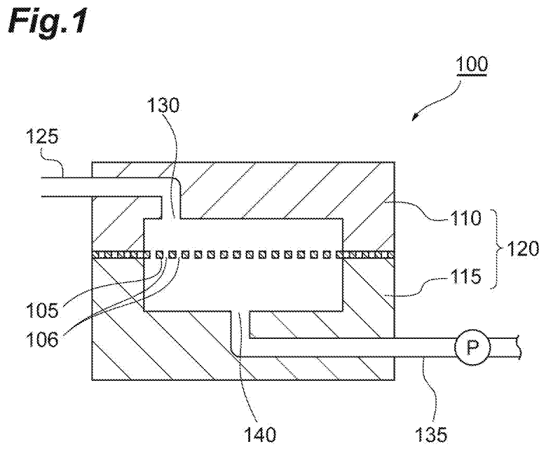

A cell trapping device to which a cell trapping filter according to an embodiment of the present invention applied will be described with reference to FIG. 1. As shown in FIG. 1, the cell trapping device 100 comprises a housing 120 including an inlet 130 connected to an inflow pipe 125 (introduction flow channel) into which a sample solution containing rare cells as a trapping target flows and an outlet 140 connected to an outlet pipe 135 (discharge flow channel) from which a sample solution flows out; and a filter 105 (cell trapping filter) disposed inside the housing 120 and having a trapping region for trapping rare cells contained in a sample solution.

The term “rare cell” as a target of trapping by the filter 105 in the cell trapping device 100 indicates a cell of a specific type contained in a biological sample. Examples of the rare cell include blood circulating tumor cells (CTCs) contained in blood and circulating endothelial cells (CECs). The CTCs are cancer cells in blood. The CECs are endothelial cells of blood vessels and become mature cells detached from the blood vessel wall by metabolism. It is said that CTCs increase in pathological conditions such as cardiovascular diseases (e.g., myocardial infarction). The cell trapping device 100 has a function of selectively capturing rare cells of a specific type contained in a sample solution, such as CTCs and CECs. When CTCs or CECs are a trapping target, blood or a solution prepared by adding an additive, such as a buffer solution, to blood can be used as a sample solution, but the sample is not limited thereto. For example, when detection of seeded and micrometastasized cancer cells is a purpose, it is also possible to use a body fluid, such as lymph, and a solution containing a body fluid as a sample solution. In addition, before the trapping of rare cells by the cell trapping device 100, the rare cells as a trapping target contained in the sample solution may be subjected to modification such as labeling.

The housing 120 is a member for holding the filter 105 for trapping rare cells and is composed of an upper member 110 and a lower member 115. The shape of the housing 120 may be, for example, a rectangular parallelepiped or a cylinder, and there is no particular restriction.

The upper stream of the inflow pipe 125 is connected to, for example, a container or piping related to a treatment solution, such as a sample solution or a cleaning solution. In addition, the lower stream of the outlet pipe 135 is connected to a pump P, and a sample solution or the like is supplied to the inside of the housing 120 from the inflow pipe 125 by operation of the pump P. In addition, a sample solution or the like is discharged to the outside from the outlet pipe 135 by operation of the pump P.

The filter 105 is provided with a plurality of through-holes 106. In the case of introducing blood, as a sample solution, into the cell trapping device 100, although red blood cells and so on of the blood pass through the through-holes 106, rare cells as a trapping target cannot pass through the through-holes 106 and stay on the surface of the filter 105. Consequently, rare cells in blood can be collected. Incidentally, it is also possible to perform additional treatment, such as staining, for the cells trapped by the filter 105. In such a case, it is possible to perform a variety of types of treatment by allowing an additional solution, such as a buffer solution, a staining solution, or a culture medium, to pass through the filter 105 trapping cells.

The shape of the cell trapping device 100 is not limited to those mentioned above. The specific configuration of the cell trapping device 100 is not particularly limited as long as it is possible to allow a sample solution containing rare cells as a trapping target to pass through a plurality of through-holes 106 provided to the filter 105.

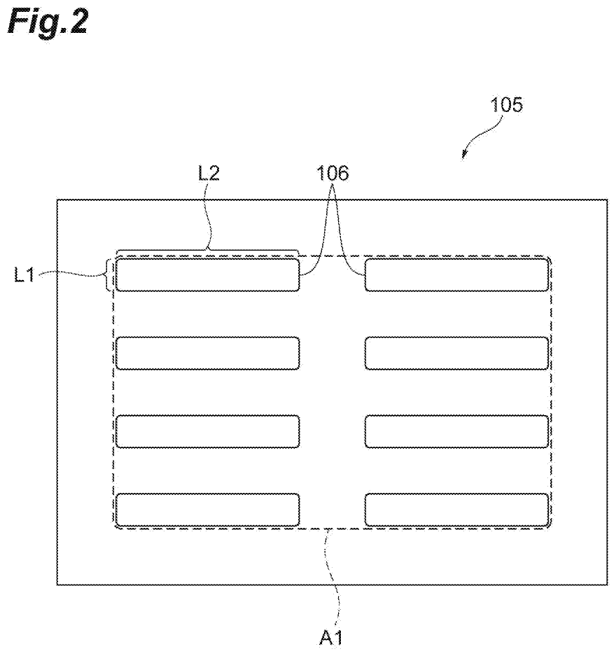

The filter 105 will be further described. FIG. 2 is a diagram schematically illustrating the upper surface of the filter 105. The filter 105 used in the cell trapping device 100 is characterized in that a palladium plating layer containing palladium as a main component is formed on the outside of the sheet-like body portion containing nickel or copper as a main component and provided with a plurality of through-holes in the thickness direction and that a gold plating layer containing gold as a main component is formed on the outside of the palladium plating layer. That is, in the filter 105, the surface of the body portion is covered with the palladium plating layer, and the surface of the palladium plating layer is covered with the gold plating layer. In the present embodiment, a definition that a specific material is “main component” means that the content of the material is 50 mass % or more.

The filter 105 has the following advantages by the main component of the body portion being nickel or copper. First of all, since nickel or copper is excellent in processability, it is possible to enhance the processing accuracy of the filter. Consequently, it is possible to further improve the capturing rate of a component as a capturing target. In addition, since a metal is rigid compared with other materials such as plastics, the size or shape is maintained even if a force is applied from the outside. Consequently, it is possible to deform blood components (in particular, white blood cells) slightly larger than the through-holes to pass therethrough and to perform highly precise separation and concentration.

The thickness of the filter 105 is preferably 3 to 50 μm, more preferably 5 to 40 μm, and particularly preferably 5 to 30 μm. If the thickness of the filter 105 is less than 3 μm, the strength of the filter 105 is decreased, which may cause a difficulty in handling property. In contrast, if the thickness is larger than 50 μm, a productivity reduction due to a longer processing time, a disadvantage in cost due to excessive material consumption, or a difficulty in microprocessing itself are concerned.

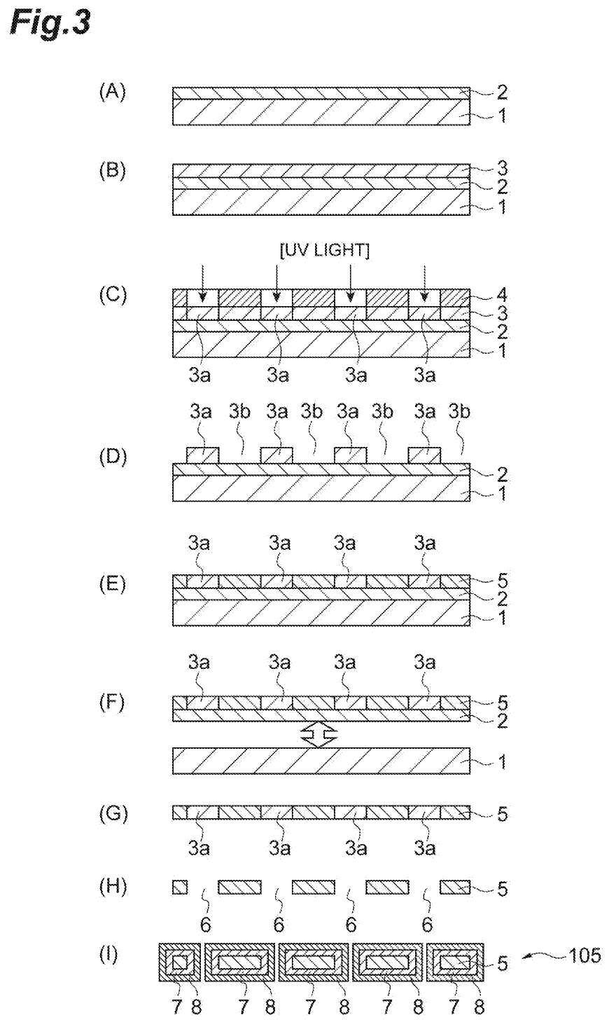

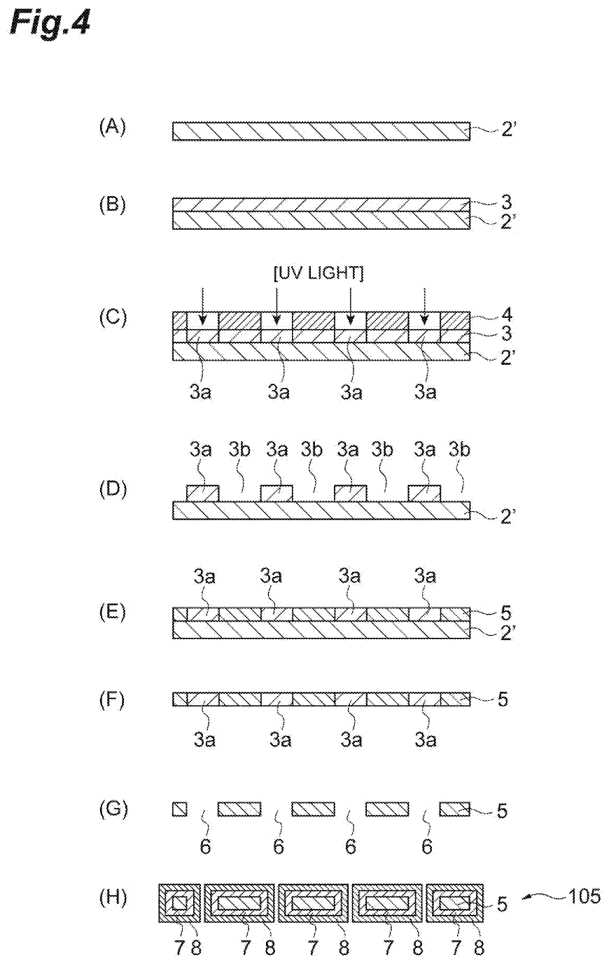

In the filter 105, it is preferable that the thickness of the palladium plating layer (in FIGS. 3 and 4, shown as palladium plating layer 7) be 0.1 to 1 μm. In addition, it is preferable that the thickness of the gold plating layer (in FIGS. 3 and 4, shown as gold plating layer 8) formed on the outside of the palladium plating layer be 0.5 to 0.8 μm. It is possible to further prevent the flow-out (elution) of metal ions of the body portion of the filter 105 covered with the palladium plating layer by controlling the palladium plating layer within the above-mentioned range. In addition, it is possible to further reduce the influence on the cells by the metal of the body portion of the filter by controlling the thickness of the gold plating layer within the above-mentioned range.

Examples of the opening shape of the plurality of the through-holes 106 provided to the filter 105 include a circle, an ellipse, a rounded rectangle, a rectangle, a square, and a waveform. FIG. 2 shows an example in which the opening shape of the through-holes 106 is a rounded rectangle. Although FIG. 2 shows an example where rounded rectangular through-holes 106 are arranged along one direction, arrangement and other factors are not particularly limited. From the viewpoint of efficiently capturing cancer cells, a circle, a rounded rectangle, a rectangle, or a waveform is preferred. In addition, from the viewpoint of preventing clogging of the filter 105, a rounded rectangle or a rectangular is particularly preferred.

The pore size and other properties of the through-holes 106 of the filter 105 are set according to the size of rare cells as a trapping target. In addition, in the through-holes 106 having an opening shape other than a circle, such as an ellipse, a rectangle, or a waveform, the pore size means the maximum value of the diameters of spheres that can pass through the respective through-holes 106 without being deformed. That is, the pore size of a through-hole 106 is, for example, when the opening shape is a rectangle or a rounded rectangle, the length of a short side of the rectangle or rounded rectangle. In the through-holes 106 of the filter 105 shown in FIG. 2, the side L1 corresponds to the short side, and the side L2 corresponds to the long side.

When the cells as a trapping target are blood circulating tumor cells (CTCs), the pore size of the through-holes 106 of the filter 105 is, at the entrance side of the filter 105, preferably 5 to 100 μm, more preferably 5 to 50 μm, and particularly preferably 5 to 30 μm. Herein, the entrance side of the through-holes 106 is the inflow side when a sample such as blood passes through the filter 105. When the cells as a trapping target are circulating endothelial cells (CECs), the pore size of the through-holes 106 of the filter 105 is, at the entrance side of the filter 105, preferably 5 to 100 μm, more preferably 5 to 50 μm, and particularly preferably 5 to 30 μm.

In addition, a cross-sectional shape of the through-hole 106 may be a shape in which the pore size at the central portion of the through-hole is larger than the pore size of the narrowest portion on the discharge side (a shape having an expanded central portion), as long as the pore size of the narrowest portion on the discharge side is equal to or larger than the pore size of the narrowest portion on the entrance side.

The average opening-rate of the through-holes 106 of the filter 105 is preferably 5% to 50%, more preferably 10% to 40%, and particularly preferably 10% to 30%. The opening rate of the through-holes 106 in the filter 105 refers to, as shown in FIG. 2, the rate of the area occupied by the through-holes 106 in the area A1 of the whole region where a sample solution actually passes in the filter 105. As shown in FIG. 1, since the peripheral portion of the filter 105 is held by the upper member 110 and the lower member 115, a sample solution actually does not pass through the through-holes 106 in the peripheral portion of the filter 105 in some cases. Accordingly, calculation of the opening rate is performed by defining the region where a sample solution actually passes and calculating the rate of the through-holes 106 occupying the resulting region μl. In addition, although a larger average opening-rate is preferred from the viewpoint of preventing clogging, an average opening-rate of higher than 50% may reduce the strength of the filter 105 or may make processing difficult. In contrast, an average opening-rate of smaller than 5% is apt to cause clogging and may reduce the rare-cell concentration performance by the filter 105.

In the present embodiment, using the cell trapping device 100 and allowing a sample solution to pass through the filter 105 is called filtration. Cells, such as rare cells, that cannot pass through the through-holes 106 by filtration remain on the filter 105. Consequently, it is possible to selectively trap specific cells with the filter 105.

Next, a method for producing the filter 105 will be described.

A method for producing the filter 105 (cell trapping filter) according to the present embodiment includes a lamination step of laminating a photosensitive resin composition on a copper substrate to form a photosensitive resin composition layer; an exposure step of exposing a predetermined portion of the photosensitive resin composition layer to active light to form a cured product of the photosensitive resin composition; a development step of removing, by development, the photosensitive resin composition layer in the portion other than the cured product of the photosensitive resin composition to form a resist pattern made of the cured product of the photosensitive resin composition on the copper substrate; a base metal plating step of performing base metal plating on the copper substrate on which the resist pattern is formed; a dissolution step of removing the copper substrate by chemical dissolution to obtain a structure consisting of the resist pattern and the base metal plating layer; a peeling step of removing the resist pattern from the structure to obtain the base metal plating layer; and a noble metal plating step of performing noble metal plating on the base metal plating layer after the peeling step.

FIG. 3, (A) to (I), is a process diagram for explaining an embodiment of a method for producing a filter 105. In this embodiment, a case in which peelable copper foil is used as a copper substrate and the body portion of the filter 105 contains nickel as the main component is described.

FIG. 3(A) shows a carrier layer 1 and peelable copper foil consisting of a copper foil layer 2. The lamination step shown in FIG. 3(B) laminates a photosensitive resin composition on the copper foil layer 2 and form a photosensitive resin composition layer 3. Subsequently, the exposure step shown in FIG. 3(C) irradiates the photosensitive resin composition layer 3 with active light (such as UV light) through a photomask 4 for photocuring the portion exposed to the light to form a cured product 3a of the photosensitive resin composition. Subsequently, the development step shown in FIG. 3(D) removes the photosensitive resin composition layer 3 in the portion 3b other than the cured product 3a of the photosensitive resin composition to form a resist pattern made of the cured product 3a of the photosensitive resin composition. Subsequently, in the base metal plating step shown in FIG. 3(E) forms a base metal plating layer 5 on the copper foil layer 2 on which the resist pattern made of the cured product 3a of the photosensitive resin composition is formed. Subsequently, as shown in FIG. 3(F), the copper foil layer 2 being peelable copper foil and the carrier layer 1 are peeled from each other. Subsequently, the dissolution step shown in FIG. 3(G) removes the copper foil layer 2 by chemical dissolution. As a result, the cured product 3a of the photosensitive resin composition and the base metal plating layer 5 remain. Subsequently, the peeling step shown in FIG. 3(H) removes the resist pattern made of the cured product 3a of the photosensitive resin composition to obtain a body portion made of the base metal plating layer 5 of the filter. The body portion of the filter is provided with through-holes 6. Subsequently, the noble metal plating step shown in FIG. 3(G) forms a palladium plating layer 7 and a gold plating layer 8 on the outside of the base metal plating layer 5. Consequently, a filter 105 is obtained.

FIG. 4, (A) to (H), is a process diagram for explaining another example of a method for producing a filter 105 according to the present embodiment. The production method shown in FIG. 4 is the same as the method for producing a filter 105 shown in FIG. 4 except that a copper substrate 2′ is used instead of the peelable copper foil used in the production method shown in FIG. 3. The method for producing a filter 105 shown in FIG. 4 is the same as the above embodiment except that the step of peeling the copper foil layer 2 being peelable copper foil and the carrier layer 1 from each other shown in FIG. 3(F) is not present. In addition, since the copper substrate 2′ is thicker than the copper foil layer 2 being peelable copper foil shown in FIG. 3, in the step of removing the copper substrate 2′ by chemical dissolution in the dissolution step, larger amounts of chemical dissolving agent and time are required than those in the above embodiment.

Next, each step of the above-mentioned method for producing a filter 105 will be described in detail.

(Lamination Step)

First of all, the lamination step will be described. When the main component of the body portion of a filter is nickel, a copper substrate is used as the substrate. Consequently, it is possible to remove the substrate by chemical dissolution. Accordingly, the dissolution step described below does not damage the body portion of the filter in removal of the substrate, and it is possible to suppress deformation of the through-holes of the filter. Accordingly, it is possible to realize a high processing accuracy of the through-holes. In addition, copper is excellent in adhesion to the photosensitive resin composition and can therefore form a resist pattern even in a small adhesion area. Accordingly, it is possible to form minute through-holes. When the main component of the body portion of the filter is copper, it is preferable to use a nickel substrate as the substrate.

Although there is no particular limitation as the copper substrate as long as it is of copper or has copper on the surface, examples thereof include copper foil having a thickness of 1 to 100 μm, copper foil tape, and peelable copper foil. From the viewpoint of workability, peelable copper foil is preferred. It is possible to shorten the time for removing the copper substrate by chemical dissolution by using peelable copper foil in the dissolution step described below.

As the photosensitive resin composition, although either a negative-type or a positive-type can be used, a negative-type photosensitive resin composition is preferred. The negative-type photosensitive resin composition preferably contains at least a binder resin, a photopolymerizable compound having an unsaturated bond, and a photopolymerization initiator. In the case of using a positive-type photosensitive resin composition, since the solubility of the portion of the photosensitive resin composition layer exposed to light by irradiation with active light in a developer increases, the portion exposed to light is removed in the development step. Hereinafter, a case using a negative-type photosensitive resin composition will be described.

The thickness of the body portion of the filter to be produced is smaller than that of the photosensitive resin composition layer. Accordingly, it is necessary to form a photosensitive resin composition layer having a thickness suitable for the thickness of the body portion of a purposed filter. For example, when the thickness of the body portion is 15 μm or less, it is necessary that the thickness of the photosensitive resin composition is 15 μm or more.

Lamination of the photosensitive resin composition on the copper substrate is performed by, for example, removing the protection film of a sheet-like photosensitive element consisting of a support film, a photosensitive resin composition, and the protection film and then press-bonding the photosensitive resin composition layer of the photosensitive element to the copper substrate with heating. Consequently, a laminate consisting of a copper substrate, a photosensitive resin composition layer, and a support film laminated in this order is obtained.

From the viewpoint of adhesion and followability, it is preferable to perform this laminating operation under reduced pressure. Although there are no particular limitations in the conditions such as heating temperature and pressure for the photosensitive resin composition layer and/or the copper substrate in the press-bonding, it is preferable to perform the press-bonding at a temperature of 70° C. to 130° C. and to perform at a pressure of about 100 to 1000 kPa. In addition, in order to improve the lamination properties in the press-bonding of the photosensitive resin composition layer, the copper substrate may be subjected to preheating treatment.

(Exposure Step)

Next, the exposure step will be described. A predetermined portion of the photosensitive resin composition layer on the copper substrate is irradiated with active light for photocuring the portion exposed to light and thereby form a cured product of the photosensitive resin composition. On this occasion, when the support film present on the photosensitive resin composition layer has transparency to active light, irradiation with active light through the support film is possible. In contrast, when the support film has an active light shielding property, the photosensitive resin composition layer is irradiated with active light after the support film is removed.

Examples of the exposure method include a method (mask exposure method) in which active light is irradiated on an image through a negative or positive mask pattern called artwork. Alternatively, a method in which active light is irradiated on an image by a direct imaging exposure method, such as a laser direct imaging (LDI) exposure method or a digital light processing (DLP) exposure method, may be employed.

As the light source of active light, it is possible to use a general light source, and examples thereof include those effectively radiating ultraviolet light, visible light, or the like, such as a carbon arc lamp, a mercury vapor arc lamp, a high-pressure mercury lamp, a xenon lamp, a gas laser such as an argon laser, a solid laser such as a YAG laser, and a semiconductor laser.

The wavelength (exposure wavelength) of active light is preferably within a range of 350 to 410 nm and more preferably within a range of 390 to 410 nm.

(Development Step)

Next, the development step will be described. The development step removes the photosensitive resin composition layer in the portion other than the cured product of the photosensitive resin composition on the copper substrate to form a resist pattern made of the cured product of the photosensitive resin composition on the copper substrate. When a support film is present on the photosensitive resin composition layer, the support film is removed, and the removal (development) of the portion other than the cured product of the photosensitive resin composition is then performed. Although the development method includes wet development and dry development, wet development is widely employed.

In the case of wet development, development is performed by a general development method using a developer corresponding to the photosensitive resin composition. Examples of the development method include those using a dip system, a paddle system, a spray system, brushing, slapping, scraping, or dipping with shaking, but a high-pressure spray system is most suitable from the viewpoint of improving the resolution. The development may be performed by a combination of two or more methods.

Examples of the developer include alkaline aqueous solutions, aqueous developers, and organic solvent developers. When an alkaline aqueous solution is used as a developer, it is safe and stable and has good operability. As the base of the alkaline aqueous solution, for example, an alkali metal hydroxide, such as a hydroxide of lithium, sodium, or potassium; a carbonate or bicarbonate of lithium, sodium, potassium, or ammonium; an alkali metal phosphate, such as potassium phosphate or sodium phosphate; or an alkali metal pyrophosphate, such as sodium pyrophosphate or potassium pyrophosphate, is used.

The alkaline aqueous solution is preferably, for example, a 0.1 to 5 mass % sodium carbonate dilute solution, a 0.1 to 5 mass % potassium carbonate dilute solution, a 0.1 to 5 mass % sodium hydroxide dilute solution, or a 0.1 to 5 mass % sodium tetraborate dilute solution. The pH of the alkaline aqueous solution is preferably within a range of 9 to 11, and the temperature is adjusted depending on the alkali developability of the photosensitive resin composition layer. The alkaline aqueous solution may contain, for example, a surfactant, a defoaming agent, and a small amount of an organic solvent for promoting the development.

After the formation of the resist pattern made of the cured product of the photosensitive resin composition on the copper substrate by removing the portion other than the cured product of the photosensitive resin composition by development, the resist pattern may be further cured, as needed, by performing heating at about 60° C. to 250° C. or exposure to light of about 0.2 to 10 J/cm².

The cross-section of the through-holes of the produced body portion has a taper shape depending on the conditions of the above-described lamination step, exposure step, and development step. Accordingly, optimization of the conditions of the lamination step, exposure step, development step is necessary in some cases. In other words, it is possible to produce a body portion having through-holes having a tapered cross-section by the above-described production method.

(Base Metal Plating Step)

The base metal plating step will be described. After the development step, base metal plating is performed on the copper substrate to form a base metal plating layer. In the case of performing base metal plating on the copper substrate, nickel plating is performed. The base metal plating layer formed by the base metal plating step finally becomes the body portion of a filter.

As described above, the material of the body portion of a filter is selected from metals containing nickel or copper as a main component.

The form of plating may be either electroplating or electroless plating. For example, as electrolytic nickel plating, examples thereof include a watt bath (nickel sulfate, nickel chloride, and boric acid are main components), a sulfamic acid bath (nickel sulfamate and boric acid are main components), and a strike bath (nickel chloride and hydrogen chloride are main components).

In electroplating, a copper substrate serves as a power feed layer, and plating deposits on the surface of copper.

In formation by electroless plating, it is preferable to degrease the surface with an acid or an alkali and so on. Subsequently, it is preferable to add a catalyst to the surface to activate the surface. As the catalyst, a noble metal, such as Pd, Au, and Pt, is mainly used.

The plating solution to be used in electroless plating may contain a complexing agent and a reducing agent, in addition to metal ions (plating component). In the case of forming a plating layer containing an alloy of nickel (Ni) as a main component by electroless plating, examples of the alloy of Ni include Ni—P, Ni—B, Ni—W, Ni—Pd, and Ni—Cu. As the reducing agent of Ni ions, for example, hypophosphorous acid or a salt thereof phosphorous acid or a salt thereof, hydrazine, borohydride, or dimethylamineborane can be used. Among these agents, electroless Ni plating using a hypophosphite is preferred. The crystallinity of Ni can be controlled from crystalline to amorphous by using a hypophosphite as the reducing agent.

The pH range of the plating solution is preferably 4.0 to 6.0 in electroplating and is preferably 4.0 to 9.0 in electroless plating. The temperature of the plating solution is preferably 40° C. to 60° C. in electroplating and is preferably 60° C. to 95° C. in electroless plating.

In both electroplating and electroless plating, it is preferable to perform cleaning and conditioning. The surface to be plated must be a clean surface. Plating on a contaminated surface causes problems, such as peeling, discoloration, or undeposition.

(Dissolution Step)

Next, the dissolution step will be described. After formation of a base metal plating layer (herein, nickel plating layer), the copper substrate is removed by chemical dissolution. Consequently, a structure consisting of the base metal plating layer becoming the body portion of a filter and the cured product of the photosensitive resin composition can be collected without performing manual work (peeling with hands). Accordingly, it is possible to produce the body portion of a filter without causing damage, such as wrinkles, bends, scratches, and curls and deformation of the minute through-holes. As the chemical dissolving agent dissolving the copper substrate, for example, Mec Bright SF-5420B (manufactured by MEC Co., Ltd.) or Copper selective etchant-CSS (manufactured by Nihon Kagaku Sangyo Co., Ltd.) can be used.

When the material of the substrate as an object to be dissolved is nickel, as the chemical dissolving agent, for example, Nickel selective etchant NC (manufactured by Nihon Kagaku Sangyo Co., Ltd.) or Mec Remover NH-1860 (manufactured by MEC Co., Ltd.) can be used.

(Peeling Step)

Next, the peeling step will be described. After the dissolution step, the resist pattern (the cured product of the photosensitive resin composition) is peeled with, for example, an alkaline aqueous solution further stronger than the alkaline aqueous solution used for development. As this strong alkaline aqueous solution, for example, it is preferable to use a 1 to 10 mass % aqueous sodium hydroxide or potassium hydroxide solution, more preferably to use a 1 to 5 mass % aqueous sodium hydroxide or potassium hydroxide solution. It is possible to collect only the base metal plating layer as the body portion of a filter by peeling the resist pattern.

Examples of the peeling system of the resist pattern include a dipping system, a spray system, and a system using ultrasonic waves, and these systems may be used alone or in combination.

(Noble Metal Plating Step: Palladium Plating)

Next, the noble metal plating step will be described. A palladium plating layer and a gold plating layer are formed by noble metal plating on the body portion prepared by the peeling step.

First of all, palladium plating is performed on the body portion. The specific method of the palladium plating is not particularly limited, and it can be performed by electroless palladium plating or electroplating. The palladium plating layer formed by electroless palladium plating is of palladium ions in the plating solution for the electroless palladium plating deposited as palladium on the surface of the body portion containing nickel as a main component by the action of a reducing agent.

The palladium plating layer 7 formed by electroless palladium plating may contain, for example, phosphorus and boron in addition to palladium. However, a palladium plating layer 7 having a purity of 99 mass % or more is preferably formed by electroless palladium plating using a formic acid compound as the reducing agent. The use of a formic acid compound can uniformly deposit a plating film of high purity particularly easily. A purity closer to 100 mass % is excellent in uniformity of the deposition form of palladium.

The palladium plating layer 7 having a palladium purity of 90 mass % or more and less than 99 mass % can be generally formed by using a plating solution containing a phosphorus-containing compound, such as hypophosphorous acid or phosphorous acid, or a boron-containing compound as a reducing agent. By using these plating solutions, a palladium-phosphorus alloy plating film or a palladium-boron alloy film is respectively formed. The concentration of the reducing agent in the plating solution, the pH, the bath temperature, and so on are adjusted such that the purity of palladium is 90 mass % or more and less than 99 mass %. Specifically, for example, when hypophosphorous acid is used as the reducing agent, it is possible to form a palladium plating layer 7 having a palladium purity of 90 mass % or more and less than 99 mass % within the range of 0.005 mol/L to 0.3 mol/L, a pH of 7.5 to 11.5, and a temperature of 40° C. to 80° C.

Alternatively, the palladium plating layer 7 may be formed by palladium electroplating. The palladium electroplating is not particularly limited as long as palladium ions in the plating solution are electrically reduced to metal palladium and palladium is deposited on the nickel surface.

(Noble Metal Plating Step: Gold Plating)

Next, a gold plating layer is formed by gold plating on the surface of the palladium plating layer 7. The gold plating can be performed electrolessly or can be performed by electrolysis. When the plating is performed by electrolysis, since the variation in thickness is large to easily affect the pore size accuracy of the filter, electroless plating is desirable.

The surface of the body portion before the gold plating may be oxidized. Accordingly, a pretreatment step of removing an oxide film may be included. The removal of an oxide film is preferably washing with an aqueous solution containing a compound forming a complex with a metal ion. Specifically, an aqueous solution containing a cyan compound, an EDTA compound, or a citric acid compound is preferred.

In particular, the citric acid compound is optimal for pretreatment of the gold plating. Specifically, the citric acid compound may be anhydride of citric acid, hydrate of citric acid, citrate, or hydrate of citrate, and specifically, for example, citric acid anhydride, citric acid monohydrate, sodium citrate, or potassium citrate can be used. The concentration thereof is preferably within a range of 0.01 to 3 mol/L, more preferably 0.03 to 2 mol/L, and particularly preferably 0.05 to 1 mol/L. By adjusting the concentration of the citric acid compound to 0.01 mol/L or more, the adhesion between the gold plating layer and the palladium plating layer is improved. If the concentration of the citric acid compound is higher than 3 mol/L, the effect is not improved, and it is economically undesirable. Dipping in a solution containing citric acid at 70° C. to 95° C. for 1 to 20 minutes is preferred.

Although the solution containing citric acid can contain a reducing agent and a buffer such as a pH adjuster that are contained in, for example, a plating solution within a range providing the effects of the invention, it is desirable that the amounts of the reducing agent, pH adjuster, etc. are small, and an aqueous solution containing citric acid only is the most preferable. The pH of the solution containing citric acid is preferably 5 to 10 and more preferably 6 to 9.

The pH adjuster is not particularly limited as long as it is an acid or an alkali, and examples of the acid include hydrochloric acid, sulfuric acid, and nitric acid; and examples of the alkali include hydroxide solutions of an alkali metal or alkaline earth metal, such as sodium hydroxide, potassium hydroxide, and sodium carbonate. As described above, the pH adjuster can be used within a range of not inhibiting the effects of citric acid. When the solution containing citric acid also contains nitric acid at a high concentration of 100 ml/L, the effect of improving the adhesive property decreases compared with the case of treating with a solution containing citric acid only.

The reducing agent is not particularly limited as long as it is reducible, and examples thereof include hypophosphorous acid, formaldehyde, dimethylamine borane, and sodium borohydride.

After the above-described pretreatment, displacement gold plating is carried out. Although the displacement gold plating includes a cyan bath and a non-cyan bath, the non-cyan bath is desirable considering the environmental load and the cytotoxicity when remained. Examples of the gold salt contained in the non-cyan bath include chloraurate, gold sulfite salt, gold thiosulfate salt, and gold thiomalate salt. The gold salts may be used alone or in combination of two or more thereof.

Gold sulfite is particularly preferred as the supply source of gold. The gold sulfite is preferably, for example, sodium gold sulfite, potassium gold sulfite, and ammonium gold sulfite.

The gold concentration is preferably within a range of 0.1 to 5 g/L. When it is less than 0.1 g/L, gold hardly deposits, and when it exceeds 5 g/L, the solution easily decomposes.

The displacement gold plating bath preferably contains ammonium salt or ethylenediamine tetraacetate salt as a complexing agent of gold. Examples of the ammonium salt include ammonium chloride and ammonium sulfate; and examples of the ethylenediamine tetraacetate salt include ethylenediamine tetraacetic acid, sodium ethylenediamine tetraacetate, potassium ethylenediamine tetraacetate, and ammonium ethylenediamine tetraacetate. The ammonium salt is preferably used within a concentration range of 7×10⁻³ to 0.4 mol/L, and if the concentration of the ammonium salt is out of this range, the solution tends to be unstable. The ethylenediamine tetraacetate salt is preferably used within a concentration range of 2×10⁻³ to 0.2 mol/L, and if the concentration of the ammonium salt is out of this range, the solution tends to be unstable.

In order to stably maintain the plating solution, it is preferred to contain 0.1 to 50 g/L sulfite. Examples of the sulfite include sodium sulfite, potassium sulfite, and ammonium sulfite.

In the case of reducing the pH by a pH adjuster, it is preferable to use chloric acid or sulfuric acid. In the case of increasing the pH, it is preferable to use sodium hydroxide, potassium hydroxide, or ammonia water. The pH is preferably adjusted to 6 to 7. In the outside of this range, the stability of the solution or the appearance of the plating is adversely affected.

It is preferable to use displacement plating at a solution temperature of 30° C. to 80° C., and in the outside of this range, the stability of the solution or the appearance of the plating is adversely affected.

Although displacement plating is carried out as described above, it is difficult to completely cover the metal by displacement plating. Accordingly, it is preferable to subsequently perform reduced electroless gold plating containing a reducing agent. The thickness of the displacement plating is preferably within a range of 0.02 to 0.1 μm. As the reduced electroless gold plating containing a reducing agent, it is possible to use a known method. The conditions for performing the reduced electroless gold plating can be appropriately changed.

The thus-formed outermost layer, gold plating layer 8, is preferably made of gold having a purity of 99 mass % or more. If the purity of gold of the gold plating layer 7 is less than 99 mass %, the cytotoxicity of the contact part increases. From the viewpoint of enhancing the reliability, it is preferable that the purity of gold of the gold plating layer 8 be 99.5 mass % or more.

(Post Treatment Step)

A post treatment step of performing surface treatment of the filter 105 produced by the above-described steps may be further performed. Specifically, it is also preferable to use a surface treatment agent containing phospholipid in a main skeleton. This hydrophobizes the filter surface and provides an effect of preventing rare cells and other cells (such as white blood cells, red blood cells, and platelets) from adhering to the surface of the filter 105. A hydrophobic layer is formed on the surface of the gold plating layer 8 of the filter 105 through the post treatment step.

As the surface treatment agent to be used in the surface treatment of the filter 105, for example, it is possible to use a biocompatible polymer. The hydrophobization treatment of the surface of the filter 105 can be performed by dipping the filter 105 in a solution containing the biocompatible polymer.

Examples of the solvent containing the biocompatible polymer include albumin of a vertebrate animal. In particular, serum albumin is desirable. Serum albumin is one of many proteins present in serum and has a molecular weight of about 66000. Although many proteins are present in serum, serum albumin accounts for about 50% to 65%.

Since albumin includes a large number of amino acids linked to one another, albumin has a large number of amino groups. An amino group forms a strong coordination bond with a noble metal (gold, platinum, or palladium). In particular, gold has almost no oxide film and therefore forms a strong bond with albumin even if a special pretreatment is not performed.

Although serum albumin of a vertebrate animal is suitably used as a surface treatment agent, among albumins, bovine serum albumin is inexpensive and is therefore preferable. In addition, among serum albumins, a fatty acid-free type has a high effect of suppressing adhesion of white blood cells, red blood cells, and platelets.

In addition, in recent years, a large number of artificial synthetic polymers that imitate living organism-derived biocompatible polymers have been synthesized. However, living cells can sense a difference between a living organism-derived biocompatible polymer and an artificial synthetic polymer. Accordingly, strictly, a living organism-derived biocompatible polymer is excellent in the effect of suppressing adhesion of cells, compared with an artificial synthetic polymer.

Although examples of the artificial synthetic polymer include silicone, a variety of polyurethanes, and polyphosphazene, a particularly excellent one is a homopolymer of 2-methacryloyloxyethyl phospholylcholine (abbreviation: MPC) or copolymer containing MPC. The structural formula thereof is shown below.

### [Chemical Formula 1]

The above-mentioned chemical formula shows an MPC polymer, wherein as R, for example, an alkyl group, hydrogen, an amino group, or a hydroxyalkyl group can be applied.

In addition, it is also possible to use commercially available MPC polymers. Examples of the commercially available MPC polymer include Lipidure-BL103, Lipidure-BL203, Lipidure-BL206, Lipidure-BL405, Lipidure-BL502, Lipidure-BL702, Lipidure-BL802, Lipidure-BL1002, Lipidure-BL1201, Lipidure-BL1301, and Lipidure-CM5206 (Lipidure is a registered trademark, all are manufactured by NOF Corporation).

The filter 105 is dipped in a diluted solution of such a biocompatible polymer. The concentration of the solution containing a biocompatible polymer is preferably within a range of 0.1% to 5.0%.

The solution for dilution is preferably an aqueous system and may contain a buffer solution of, for example, phosphoric acid. Alternatively, a blood coagulation inhibitor, such as EDTA or heparin, may be contained.

The treatment time (dipping time) is preferably 1 minute or more and 60 minutes or less and further preferably 1 minute or more and 10 minutes or less. It is possible to promote a coordination bond between a biocompatible polymer and the surface of a gold plating layer of the filter 105 by performing the treatment for 1 minute or more. In addition, it is possible to prevent lengthening of the work time by adjusting the treatment time to 60 minutes or less.

It is possible to prevent rare cells and other cells from adhering to the surface of the filter 105 and to increase the trapping efficiency of rare cells by using the filter 105 after the above-described post treatment step.

As described above, the filter 105 for trapping cells according to the present embodiment and the cell trapping device 100 utilizing the filter 105 are characterized in that the surface of the body portion (base metal plating layer 5), of the filter 105, containing nickel or copper as a main component is covered with a palladium plating layer 7 and a gold plating layer 8. Consequently, it is possible to suppress the metal of the body portion from flowing out into, for example, the sample solution.

Conventionally, it has been investigated to use a metal filter as the filter 105 for trapping cells. In addition, as the material of the filter 105, base metals, which are inexpensive among metals, are used as main components in many cases. However, in the case of using a filter of a base metal, there is a risk of elution of the metal component constituting the filter into a sample solution, in particular, it is significant when a complexing agent is added to the sample solution or the like. For example, when rare cells in blood are a trapping target, a sample solution in which EDTA for preventing coagulation of blood is mixed with blood may be used. In such a case, since the EDTA works as a complexing agent, there is a risk of elution of metal ions of the base metal having a low ionization tendency.

As the main component of a metal filter, among base metals, nickel or copper is often selected from the viewpoint of cost and processability. However, metal ions of nickel and copper have strong cytotoxicity. Accordingly, it is conceived that the metal ions eluted from a filter may damage the rare cells trapped on the filter.

In order prevent elution of metal ions of a base metal into a sample solution, for example, it is conceived to produce a filter using a noble metal. However, production of a filter from a noble metal only is not realistic in terms of cost. Accordingly, conventionally, gold plating has been applied to a filter made from a base metal.

However, it was confirmed that even in the case of applying gold plating to the surface of the body portion of a filter made from a base metal, a large amount of metal ions of the base metal flow out (elute) into a sample solution in some cases. Specifically, it was revealed that if a pinhole is formed in the gold plating of the body portion of the filter and there is a portion where the sample solution and the base metal are in contact with each other, even if the portion is only one, a large amount of metal ions of the internal body portion of a filter elute. It was revealed that if a small pinhole is formed in gold plating, even if it is very small, metal ions of the body portion flow out (elute) to the outside. In addition, it was confirmed that a pinhole occurs even when the surface of the body portion of a filter is uniformized. Accordingly, it was revealed that it is very difficult to prevent occurrence of pinholes in the step of applying gold plating to the body portion of a filter. Although a countermeasure, such as an increase in the thickness of gold plating, is also conceivable as another method for preventing formation of pinholes in the gold plating, the cost relating to the formation of a gold plating layer increases.

Accordingly, in the filter 105 for trapping cells according to the present embodiment, a palladium plating layer 7 is first formed on the surface of the body portion (base metal plating layer 5) containing nickel or copper as a main component of a filter 105, and a gold plating layer 8 is formed on the outside of the palladium plating layer 7. It is possible to prevent the body portion from exposing to the outside by that two noble metal layers are thus laminated on the surface of the body portion. Even if a pinhole remains when the outmost surface of the gold plating layer 8 is formed, since the palladium plating layer 7 is disposed on the inner side of the gold plating layer 8, it is possible to prevent the body portion on the inner side of the palladium plating layer 7 from being exposed to the outside and becoming in contact with, for example, a sample solution. Accordingly, in the filter 105 for trapping cells according to the present embodiment and the cell trapping device 100 utilizing this filter 105, it is possible to suitably perform trapping of rare cells, and it is also possible to prevent the cells trapped on the filter 105 from being damaged by flow-out of metal ions of the body portion.

In the filter 105 for trapping cells according to the present embodiment, it is possible to prevent the flow-out (elution) of metal ions of the body portion of the filter 105 by adjusting the thickness of the palladium plating layer 7 to 0.1 to 1 μm.

In the case of including a hydrophobic layer formed from a surface treatment agent containing phospholipid in a main skeleton on the surface of the gold plating layer 8 of the filter 105, it is possible to prevent rare cells and other cells from adhering to the surface of the filter 105 and to increase the trapping efficiency of rare cells.

Furthermore, in the cell trapping method using the filter 105 for trapping cells according to the present embodiment, filtration with the filter 105 selectively traps specific cells contained in a sample solution. It is possible to selectively trap specific cells, while preventing cells from, for example, being damaged by flow-out of metal ions of the body portion of the filter 105, by performing filtration using the filter 105 described in the above embodiment.

Examples of the cell observation method using the filter 105 for trapping cells according to the present embodiment include a method in which specific cells contained in a sample solution are selectively trapped by filtration with the filter 105, and the trapped cells are then fixed with a fixing solution and are observed under an electron microscope. As the cell-fixing method using a fixing solution, a known method can be used. It is possible to suitably perform observation of specific cells, while preventing cells from, for example, being damaged by flow-out of metal ions of the body portion of the filter 105, by performing filtration using the filter 105 described in the above embodiment, then fixing using a fixing solution, and observation. Although the fixing solution is not particularly limited, for example, aldehyde such as glutaraldehyde and formaldehyde or osmium teraoxide can be used.

Examples of the cell culturing method using the filter 105 for trapping cells according to the present embodiment include a method in which specific cells contained in a sample solution are selectively trapped by filtration with the filter 105 and the trapped cells on the filter 105 are then cultured in a culture medium. Specifically, it is possible to perform the culture by dipping the filter 105 in a state of trapping cells in a culture medium. In the case of performing filtration using the filter 105 described in the above embodiment and then culturing the cells in a culture medium on the filter 105, it is possible to suitably perform the culture of specific cells, while preventing cells from, for example, being damaged by flow-out of metal ions of the body portion of the filter 105.

The present invention has been described in detail based on the embodiments. However, the present invention is not limited to the above embodiments. The present invention can be variously modified as below within a scope which does not depart from the gist of the invention.

For example, in the above embodiment, an example in which the palladium plating layer 7 and the gold plating layer 8 are respectively formed by plating on the outer surface of the body portion (base metal plating layer 5) of the filter 105 has been explained. However, in the filter 105 according to an aspect of the present invention, as long as the outer surface of the body portion containing nickel or copper as a main component is covered with a palladium layer and a gold layer, the methods for forming the palladium layer and the gold layer are not particularly limited. Accordingly, the filter 105 may be produced by laminating a palladium layer and a gold layer on the outside of the body portion of the filter 105 using a technique (for example, sputtering, vapor deposition, or chemical vapor deposition (CVD)) different from plating.

### EXAMPLES

<Preparation of Filter>

### Example 1

A photosensitive resin composition (PHOTEC (registered trademark) RD-1225, thickness: 25 in, manufactured by Hitachi Chemical Company, Ltd.) was laminated on a copper foil layer of a 250-mm square substrate (MCL (registered trademark)-E679F: peelable copper foil having a copper foil layer on a carrier layer, manufactured by Hitachi Chemical Company, Ltd.). The lamination was performed under conditions of a roll temperature of 90° C., a pressure of 0.3 MPa, and conveyor speed of 2.0 m/min.

Subsequently, a glass mask was left to stand on the photosensitive resin composition layer of the substrate. The glass mask included light transmissive portions that were rectangles oriented in the same direction and were aligned at constant pitches in the major and minor axis directions, where the size of each rectangle was 8×30 μm, and the pitches in the major and minor axis directions were both 60 μm. Subsequently, in a vacuum of 600 mmHg or less, ultraviolet light was irradiated at an exposure amount of 40 mJ/cm² with an ultraviolet irradiation apparatus irradiating parallel light from the top of the substrate carrying the glass mask.

Subsequently, development was performed using a 1.0% aqueous sodium carbonate solution at a temperature of 30° C., a spray pressure of 0.1 MPa, and a development time of about 30 seconds to remove the photosensitive resin composition layer other than the exposure portion for forming a resist pattern, of the cured product of the photosensitive resin composition, in the form of rectangles vertically standing on the substrate.

Subsequently, plating was performed in a nickel plating solution adjusted to a pH of 4.5 at a temperature of 55° C. for about 20 minutes to form a nickel plating layer. The composition of the nickel plating solution is shown in Table 1. After the initial make-up of electrolytic bath, additive A (product name: NSF-H2, manufactured by Nihon Kagaku Sangyo Co., Ltd.) for electrolytic nickel plating was added.

TABLE 1

| Composition of plating solution | Concentration (g/L) |
|---|---|
| Nickel sulfamate | 450 |
| Nickel chloride | 5 |
| Boric acid | 30 |

The resulting nickel plating layer was peeled off together with the peelable copper foil of the substrate. Subsequently, the copper substrate (peelable copper foil) and copper were removed by stirring treatment in a chemical dissolving agent (Mec Bright SF-5420B, manufactured by MEC Co., Ltd.) at a temperature of 40° C. for about 120 minutes. Consequently, a structure consisting of a base metal plating layer and a cured product of a photosensitive resin composition was collected.

Subsequently, the cured product (resist patter) of the photosensitive resin composition in the structure was removed by sonication in a resist peeling liquid (P3 Poleve, manufactured by Henkel) at a temperature of 55° C. for about 60 minutes to produce a base metal filter corresponding to the body portion of a filter.

After that, noble metal plating treatment was performed on the surface of the base metal filter to form a palladium plating layer and a gold plating layer. The conditions for the noble metal plating treatment were as follows:

Degreasing: Z-200 (manufactured by World Metal Co., Ltd., trade name), 60° C., 1 minute;

Washing with water: room temperature, 2 minutes;

Electroless palladium plating: palladium plated coating thickness: 0.5 μm, HPS-3000 (manufactured by Hitachi Chemical Company, Ltd., trade name): 70° C., 5 minutes;

Washing with water: room temperature, 2 minutes;

Displacement gold plating: gold plated coating thickness: 0.02 μm, HGS-100, (manufactured by Hitachi Chemical Company, Ltd., trade name): 85° C., 10 minutes;

Washing with water: room temperature, 2 minutes; and

Drying: 85° C., 30 minutes.

After that, hydrophobization treatment of the surface of the filter was performed to form a hydrophobic layer on the surface. Specifically, the hydrophobization treatment of the surface was performed using a solution prepared by diluting a biocompatible polymer (Lipidure (registered trademark)-CM5206, manufactured by NOF Corporation) to 0.5 wt % with 98 wt % ethanol as a surface treatment agent and by dipping the filter after noble metal plating in the surface treatment agent. As described above, a filter according to Example 1 was obtained.

### Example 2

A filter according to Example 2 was produced in the same manner as Example 1, except that, compared with Example 1, the hydrophobization treatment of the surface in the noble metal plating treatment was not carried out.

### Example 3

A filter according to Example 3 was produced in the same manner as Example 1, except that, compared with Example 1, the additive for the electrolytic nickel plating was changed from additive A to additive B (product name: NSF-H2, manufactured by Nihon Kagaku Sangyo Co., Ltd.), and the hydrophobization treatment of the surface was not carried out.

### Comparative Example 1

A filter according to Comparative Example 1 was produced in the same manner as Example 1, except that, compared with Example 1, the electroless palladium plating step and the hydrophobization treatment of the surface in the noble metal plating treatment were not carried out.

### Comparative Example 2

A filter according to Comparative Example 2 was produced in the same manner as Example 1, except that, compared with Example 1, the additive for the electrolytic nickel plating was changed from additive A to additive B (product name: NSF-H2, manufactured by Nihon Kagaku Sangyo Co., Ltd.), and the electroless palladium plating step and the hydrophobization treatment of the surface in the noble metal plating treatment were not carried out.

### Comparative Example 3

A filter according to Comparative Example 3 was produced in the same manner as Example 1, except that, compared with Example 1, the additive for the electrolytic nickel plating was changed from additive A to additive B (product name: NSF-H2, manufactured by Nihon Kagaku Sangyo Co., Ltd.), and the electroless palladium plating step in the noble metal plating treatment was not carried out.

<Evaluation 1: Evaluation of State of Elution>

The state of elution of a base metal to the periphery of a filter was measured by an atomic absorption measurement method. Specifically, the filters according to Examples 1 to 4 and Comparative Examples 1 to 3 were put in a 6-well plate, and 1 mL of a wash buffer (phosphate buffer containing 0.37 g/L of EDTA, pH 7.4) was added to each well, followed by dipping for 3 hours. Subsequently, the wash buffer was diluted to 10 mL with pure water. Subsequently, the concentration of nickel in the diluted solution was measured with a polarized Zeeman atomic absorption spectrophotometer (model No.: Z-5310, manufactured by Hitachi High-Technologies Corporation). The results of the measurement are shown in Table 2.

TABLE 2

| | Filter material | Additive | Ni concentration (ppm) |
|---|---|---|---|
| Example 1 | Ni/Pd/Au + MPC | A | 0.04 |
| Comparative | Ni/Au | A | 1.18 |
| Example 1 | | | |
| Example 2 | Ni/Pd/Au | A | 0.04 |
| Comparative | Ni/Au | B | 0.73 |
| Example 2 | | | |
| Example 3 | Ni/Pd/Au | B | 0.07 |
| Comparative | Ni/Au + MPC | B | 0.96 |
| Example 3 | | | |

It was demonstrated that the condition of the surface of a nickel plating layer becoming the body portion of a filter is changed by changing the type of the additive from additive A to additive B. In the case of additive A, a plating layer having a flat surface was formed, and in the case of additive B, a semi-glossy plating surface was formed. However, it was demonstrated from the results shown in Table 2 that even in different conditions of surfaces, equivalent results were obtained.

In addition, according to the results of Table 2, it was demonstrated that also in the case of forming a hydrophobic layer containing a biocompatible polymer on the surface of the gold layer, the concentration of nickel in the diluted solution was prevented from increasing. Accordingly, it was demonstrated that it is possible to prevent flow-out (elution) of metal ions of the body portion of a filter even in the case of stacking a layer for improving the function on the outside of the gold layer.

In addition, the evaluation was performed under the same conditions as in Examples 1 to 3 and Comparative Examples 1 to 3 described above by changing the main component of the filter material to copper (Cu), and it was demonstrated that elution of Cu is suppressed by providing a palladium layer.

Table 3 shows the results of measurement with an atomic absorption spectrophotometer. As shown in Table 3, it was demonstrated that the elution amount of nickel is small when a palladium layer is present. In addition, in Table 3, the results of the wash buffer alone (Reference 1) measured with an atomic absorption spectrophotometer are also shown as reference. Reference 1 shows conditions not using a filter.

TABLE 3

| | | Atomic absorption | | | | | |
|---|---|---|---|---|---|---|---|
| | | spectrophotometer | | | | Survival | Culture |
| | Filter | measured value (ppm) | | | | rate | on |
| | material | Ni | Cu | Pd | Au | (%) | filter |
| Example 1 | Ni/Pd/Au + | 0.04 | 0.15 | 0.02 | 0.21 | ○ | OK |
| | MPC | | | | | | |
| Example 2 | Ni/Pd/Au | 0.04 | 0.15 | 0.01 | 0.19 | ○ | OK |
| Example 3 | Ni/Pd/Au | 0.07 | 0.15 | 0.01 | 0.1 | ○ | OK |
| Com- | Ni/Au | 1.18 | 0.15 | 0.01 | 0.17 | × | NG |
| parative | | | | | | | |
| Example 1 | | | | | | | |
| Com- | Ni/Au | 0.73 | 0.15 | 0.01 | 0.08 | × | NG |
| parative | | | | | | | |
| Example 2 | | | | | | | |
| Com- | Ni/Au + MPC | 0.96 | 0.15 | 0.02 | 0.15 | × | NG |
| parative | | | | | | | |
| Example 3 | | | | | | | |
| Reference | Wash buffer | 0 | 0.15 | 0.01 | 0 | ○ | — |
| 1 | (without filter) | | | | | | |
| Reference | Wash buffer | — | — | — | — | × | — |
| 2 | containing Ni | | | | | | |
| | (1 ppm) | | | | | | |
| | (without filter) | | | | | | |

<Evaluation 2: Cell Attribute>

Filters produced by the same processes as in Examples 1 to 3 and Comparative Examples 1 to 3 were each dipped in a wash buffer to allow the metals to elute, and the cytotoxicity of each eluate was investigated. As reference, a wash buffer (Reference 1) in which no filter was dipped was prepared as a negative control, and a wash buffer (Reference 2) containing Ni (1 ppm) was prepared as a positive control.

(Test Method)

1. First of all, each of the filters and 1 mL of a wash buffer were put in a 6-well dish, and after dipping for 3 hours, only the filters were taken out. In Reference 1 and Reference 2, the same procedure was carried out without putting a filter.
2. Cultured human bronchiole-alveolar cancer cells (NCI-H358) were added at about 100000 cells per well.
3. Incubation was performed in a 5% CO₂ incubator (37° C.) for 30 minutes.
4. 100 μL of 0.25% Trypsin EDTA (manufactured by FUJIFILM Wako Pure Chemical Corporation) was added for peeling the cells adhered to the bottom of each well.
5. After 3 minutes, 100 μL of a cell culture medium (RPMI-1640, manufactured by FUJIFILM Wako Pure Chemical Corporation) was added to each well to terminate the reaction of trypsin.
6. After staining with trypan blue, living cells and dead cells were counted using a cell counting board.

(Results)

The survival rates are shown in Table 3. A case having a survival rate of 50% or more is indicate by “◯”, and a case having a survival rate of less than 50% is indicated by “×”. As shown in Table 3, it was demonstrated that there is a correlation between the elution amount of Ni and the survival rate of cells, and it was revealed that the survival rate decreases as the elution amount of Ni increases. In contrast, it was demonstrated that in the case of using a filter including a palladium layer, the survival rate of cells is high.

<Evaluation 3: Culture Availability>

If concentrated cells can be cultured on a filter, it is possible to expect development in various fields in the future, and the possibility of the culture was therefore investigated.

Filters prepared by the same processes as in Examples 1 to 3 and Comparative Examples 1 to 3 were each dipped in a culture medium, and cells were cultured therein.

(Test Method)

1. Cultured human alveolar epithelial adenocarcinoma cells A549 were stained with Cell Tracker Green and were diluted with a cell culture medium (RPMI-1640, manufactured by FUJIFILM Wako Pure Chemical Corporation) such that the number of cells was 5×10⁴ cells/mL.
2. Each of the filters prepared by the same processes as in Examples 1 to 3 and Comparative Examples 1 to 3 and 2 mL of the above-mentioned cell solution were put in a 6-well dish, followed by culturing in a 5% CO₂ incubator at 37° C. for 2 days.
3. The cells remained in the dish were observed using an inverted microscope and a fluorescence microscope (Axio Imager 2, manufactured by Zeiss). Images of the observation field of view were acquired, and the number of cancer cells trapped by the filter was counted.

(Results)

Table 3 shows whether the culture is possible or not. As shown in Table 3, it was demonstrated that culture of cells is possible on a filter including a palladium layer.

### REFERENCE SIGNS LIST

1: MCL, 2: copper foil layer, 2′: copper substrate, 3: photosensitive resin composition layer, 3a: cured product of photosensitive resin composition, 4: photomask, 5: base metal plating layer (body portion), 6: through-hole, 7: palladium plating layer, 8: gold plating layer, 100: cell trapping device, 105: filter, 110: upper member, 115: lower member, 120: housing

1. A cell trapping filter comprising:
a sheet-like body portion containing nickel or copper as a main component and provided with a plurality of through-holes in a thickness direction;
a palladium layer containing palladium as a main component and covering a surface of the body portion; and
a gold layer containing gold as a main component and covering a surface of the palladium layer.

2. The cell trapping filter according to claim 1, wherein the palladium layer has a thickness of 0.1 to 1 μm.

3. The cell trapping filter according to claim 1, further comprising:
a hydrophobic layer formed on a surface of the gold layer by a surface treatment agent having phospholipid in part of a skeleton.

4. A cell trapping device comprising:
a housing including an introduction flow channel for introducing a sample solution into the inside and a discharge flow channel for discharging the sample solution to the outside; and
a cell trapping filter according to claim 1 disposed on a flow channel inside the housing between the introduction flow channel and the discharge flow channel in such a manner that the sample passes through the through-holes.

5. A cell trapping method comprising selectively trapping a specific cell contained in a sample solution by performing filtration with a cell trapping filter according to claim 1.

6. A cell observation method comprising selectively trapping a specific cell contained in a sample solution by performing filtration with a cell trapping filter according to claim 1, fixing the specific cell trapped on the cell trapping filter with a fixing solution, and then observing the cell under an electron microscope.

7. A cell culturing method comprising selectively trapping a specific cell contained in a sample solution by performing filtration with a cell trapping filter according to claim 1 and culturing the specific cell trapped on the cell trapping filter in a culture medium.
